(19) United States
(12) Patent Application Publication
(10) Pub. No.: US 2013/0295562 A1
(43) Pub. Date: Nov. 7, 2013

(54) METHODS FOR DETECTING RISK OF MYELODYSPLASTIC SYNDROME BY GENOTYPIC ANALYSIS

(71) Applicants: Wanlong Ma, Aliso Viejo, CA (US); Maher Albitar, Coto De Caza, CA (US)

(72) Inventors: Wanlong Ma, Aliso Viejo, CA (US); Maher Albitar, Coto De Caza, CA (US)

(73) Assignee: Quest Diagnostics Investments Incorporated

(21) Appl. No.: 13/513,131

(22) PCT Filed: Oct. 29, 2010
(86) PCT No.: PCT/US10/54692

§ 371 Date: Aug. 31, 2012

Related U.S. Application Data
(60) Provisional application No. 61/265,628, filed on Dec. 1, 2009.
(60) Provisional application No. 61/266,607, filed on Dec. 4, 2009.

(51) Int. Cl. C12Q 1/68

(52) U.S. Cl. CPC C12Q 1/6886
USPC 435/6.11

(57) ABSTRACT

The present invention provides methods for detecting the risk of developing leukemia using genotyping analysis, for example of a SNP located in the promoter region of EPO. The present invention also provides kits and nucleic acids for the detection of the risk genotype.

### FIELD OF THE INVENTION

The present invention relates generally to the field of cancer diagnostics and, in particular, the diagnosis and prognosis of patients having a myeloproliferative disease.

### BACKGROUND OF THE INVENTION

The following description of the background is provided to aid in the understanding of the invention and is not an admission of prior art.

Myelodysplastic syndrome (MDS) is a category of hematopoietic disorders characterized by the insufficiency of one or more types of blood cells due to abnormal production by the hematopoietic stem cells in bone marrow. The stem cells continue to divide, but the failure of these cells to differentiate result in the accumulation of undifferentiated primitive blood cells, myeloblasts, in the bone marrow without adequate production of the mature blood cells needed in the circulatory system. This disorder is characterized by neutropenia, anemia and/or thrombocytopenia, and changes in the spleen and liver are also occasionally seen in patients with MDS. In addition to the morbidity and mortality associated with these complications of MDS, the disease progresses to acute myelogenous leukemia (AML) in approximately 30% of MDS patients.

The risk of developing MDS is greatly increased by environmental factors, such as exposure to carcinogens and radiation. Secondary MDS develops after cancer treatments with radiation and radiomimetics. It is thought that such toxins causes genetic damage in the stem cells, resulting in the dysregulation of hematopoietic stem cells. While numerous toxic agents have been associated with the risk of MDS, genetic factors for susceptibility have not been well defined.

The role of the underlying genetic background in developing MDS and its prognosis has been appreciated, but it is not yet fully understood. For example, in some MDS patients, chromosomal genotyping reveals a deletion in the 5q arm. This region has been associated with disordered hematopoiesis, and certain treatments, such as lenalidomide, have been shown to be more effective in those MDS patients having that deletion. See for example, Bunn H F (1986) Clinics in Haematology 15 (4): 1023-35; List A, Dewald G, Bennett J, et al. (2006) N. Engl. J. Med. 355 (14): 1456-65. Additionally, drugs that target methylation of the DNA have been shown to be effective in some patients, particularly those with advanced disease, but overall response remains low due to heterogeneity in disease causation. Kantarjian H., et al., (2007) Blood 109(1):52-7. Accordingly, there remains a need for identifying the specific underlying genetic etiologies for this multi-origin disease.

Genotypic analysis using single nucleotide polymorphisms (SNPs) has been used for measuring and tracking allelic frequency and heritance in populations, as well as reference points for assembly contigs for genomic mapping. More recently SNPs have been used to identify alleles associated with disease risk.

### SUMMARY OF THE INVENTION

The present invention is based on the identification that individuals homozygous for the G allele (i.e., homozygous for the G allele at SNP rs1617640 of the EPO promoter), corresponding to nucleotide 27 of SEQ ID NO: 1, have an increased risk of developing a myeloproliferative disease and/or have a poor prognosis relative to individuals that are heterozygous (G/T genotype) or homozygous wildtype (T/T genotype).

In one aspect, the invention provides a method of determining a prognosis for a subject diagnosed with leukemia comprising: i) determining the zygosity status of the subject at the nucleotide corresponding to SNP rs1617640 in the erythropoietin gene promoter; and ii) identifying the subject as having a poor prognosis when the zygosity status is homozygous G/G. Optionally, the prognosis based on the zygosity status of SNP rs1617640 may be determined in conjunction with other clinical factors. In some embodiments, the poor prognoses include, for example, shorter survival time, shorter complete remission duration, and shorter event-free survival. Preferably, the poor prognosis is a shorter complete remission duration.

In another aspect, the invention provides a method of identifying a subject at risk of developing leukemia comprising: i) determining the zygosity status of the subject at the nucleotide corresponding to SNP rs1617640 in the erythropoietin gene promoter; and ii) identifying the subject as having an increased risk of leukemia when the zygosity status is the homozygous G/G genotype. Optionally, the increased risk of leukemia based on the zygosity status of SNP rs1617640 may be determined in conjunction with other clinical factors.

In any of the foregoing aspects of the invention, the genotype may be assessed using any convenient nucleic acid obtained from the individual (e.g., genomic nucleic acid) which may be obtained from any suitable biological sample (e.g., whole blood, serum, plasma, biopsy sample, or other tissue sample).

The leukemia for which a diagnosis or prognosis may be determined include, for example, myelodysplastic syndrome (MDS), acute lymphoblastic leukemia (ALL), acute myeloid leukemia (AML), chronic lymphocytic leukemia (CLL), and chronic myeloid leukemia (CML). Preferably, the leukemia is MDS or ALL.

Suitable methods for assessing the zygosity status in any of the foregoing methods include, for example, nucleic acid sequencing, probe hybridization, and a primer extension reaction.

The term “SNP rs1617640” as used herein means the nucleotide in the human EPO gene promoter which corresponds to the nucleotide at position 27 of SEQ ID NO: 1 which is found to be a thymine (T) in wildtypes and a guanine (G) in the mutant SNP. The sequence of SEQ ID NO: 1 is merely exemplary of the relevant region of the EPO gene promoter and the artisan understands that other sequence variations are possible for nucleotides at positions other than position 27.

The term “myeloproliferative disease” as used herein means a disorder of a bone marrow or lymph node-derived cell type, such as a white blood cell. A myeloproliferative disease is generally manifest by abnormal cell division resulting in an abnormal level of a particular hematological cell population. The abnormal cell division underlying a proliferative hematological disorder is typically inherent in the cells and not a normal physiological response to infection or inflammation. Leukemia is a type of myeloproliferative disease. Exemplary myeloproliferative diseases include, but are not limited to, acute myeloid leukemia (AML), acute lymphoblastic leukemia (ALL), chronic lymphocytic leukemia (CLL), myelodysplastic syndrome (MDS), chronic myeloid leukemia (CML), hairy cell leukemia, leukemic manifestations of lymphomas, multiple myeloma, polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), and unclassified myeloproliferative diseases (UMPD or MPD-NC). Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma).

The term “diagnose” or “diagnosis” or “diagnosing” as used herein refer to distinguishing or identifying a disease, syndrome or condition or distinguishing or identifying a person having a particular disease, syndrome or condition. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease.

The term “prognosis” as used herein refers to a prediction of the probable course and outcome of a clinical condition or disease. A prognosis of a patient is usually made by evaluating factors or symptoms of a disease that are indicative of a favorable or unfavorable course or outcome of the disease.

The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of years, on average that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

The term “poor prognosis” as used herein, in the context of a patient having a leukemia and the G/G genotype (i.e., homozygous for the G allele at SNP rs1617640 of the EPO promoter), refers to an increased likelihood that the patient will have a worse outcome in a clinical condition relative to a patient diagnosed as having the same disease but having the T/T genotype. A poor prognosis may be expressed in any relevant prognostic terms and may include, for example, the expectation of a reduced duration of remission, reduced survival rate, and reduced survival duration.

The term “zygosity status” as used herein refers to a sample, a cell population, or an organism as appearing heterozygous, homozygous, or hemizygous as determined by testing methods known in the art and described herein. The term “zygosity status of a nucleic acid” means determining whether the source of nucleic acid appears heterozygous, homozygous, or hemizygous. The “zygosity status” may refer to differences in a single nucleotide in a sequence. In some methods, the zygosity status of a sample with respect to a single mutation may be categorized as homozygous wild-type, heterozygous (i.e., one wild-type allele and one mutant allele), homozygous mutant, or hemizygous (i.e., a single copy of either the wild-type or mutant allele). In the context of the present invention, the zygosity status identifies whether an individual has the G/G, G/T, or T/T genotype for SNP rs1617640 of the EPO promoter (i.e., at nucleotide position 27 of SEQ ID NO: 1).

The zygosity status in a sample may be determined by methods known in the art including sequence-specific, quantitative detection methods. Other methods may involve determining the area under the curves of the sequencing peaks from standard sequencing electropherograms, such as those created using ABI Sequencing Systems, (Applied Biosystems, Foster City Calif.). For example, the presence of only a single peak such as a “G” on an electropherogram in a position representative of a particular nucleotide is an indication that the nucleic acids in the sample contain only one nucleotide at that position, the “G.” The sample may then be categorized as homozygous because only one allele is detected. The presence of two peaks, for example, a “G” peak and a “T” peak in the same position on the electropherogram indicates that the sample contains two species of nucleic acids; one species carries the “G” at the nucleotide position in question, the other carries the “T” at the nucleotide position in question. The sample may then be categorized as heterozygous because more than one allele is detected.

As used herein, the term “sample” or “biological sample” refers to any liquid or solid material obtained from a biological source, such a cell or tissue sample or bodily fluids. “Bodily fluids” include, but are not limited to, blood, serum, plasma, saliva, cerebrospinal fluid, pleural fluid, tears, lactal duct fluid, lymph, sputum, urine, saliva, amniotic fluid, and semen. A sample may include a bodily fluid that is “acellular.” An “acellular bodily fluid” includes less than about 1% (w/w) whole cellular material. Plasma or serum are examples of acellular bodily fluids. A sample may include a specimen of natural or synthetic origin. Exemplary sample tissues include, but are not limited to bone marrow or tissue (e.g. biopsy material).

As used herein, the term “specifically binds,” when referring to a binding moiety, is meant that the moiety is capable of discriminating between a various target sequences. For example, an oligonucleotide (e.g., a primer or probe) that specifically binds to a mutant target sequence is one that hybridizes preferentially to the target sequence (e.g., the wildtype sequence) over the other sequence variants (e.g., mutant and polymorphic sequences). Preferably, oligonucleotides specifically bind to their target sequences under high stringency hybridization conditions.

As used herein the term “stringency” is used in reference to the conditions of temperature, ionic strength, and the presence of other compounds, under which nucleic acid hybridizations are conducted. With high stringency conditions, nucleic acid base pairing will occur only between nucleic acids that have sufficiently long segment with a high frequency of complementary base sequences.

Exemplary hybridization conditions are as follows. High stringency generally refers to conditions that permit hybridization of only those nucleic acid sequences that form stable hybrids in 0.018M NaCl at 65° C. High stringency conditions can be provided, for example, by hybridization in 50% formamide, 5×Denhardt's solution, 5×SSC (saline sodium citrate) 0.2% SDS (sodium dodecyl sulphate) at 42° C., followed by washing in 0.1×SSC, and 0.1% SDS at 65° C. Moderate stringency refers to conditions equivalent to hybridization in 50% formamide, 5×Denhardt's solution, 5×SSC, 0.2% SDS at 42° C., followed by washing in 0.2×SSC, 0.2% SDS, at 65° C. Low stringency refers to conditions equivalent to hybridization in 10% formamide, 5×Denhardt's solution, 6×SSC, 0.2% SDS, followed by washing in 1×SSC, 0.2% SDS, at 50° C.

“Odds ratio” as used herein refers to the odds that a SNP is found in a disease patient population over the odds that it is found in a nondisease patient population. Methods for determining the odds ratio are provided herein.

### DETAILED DESCRIPTION

The present invention is based on the identification of a single nucleotide polymorphism (SNP) in the EPO promoter which is associated with certain leukemias including MDS. The gene encoding EPO resides on chromosome 7q21, while the molecular structure of the EPO protein is described in Romanowski et al., Hematol. Oncol. Clin. North Am. (1994) 8:885-894. Specifically, the EPO promoter SNP is designated rs1617640 (see, the HapMap database developed by the International HapMap Consortium) and has the following sequence:

(SEQ ID NO: 1)
5′-ATGGCTTCTG GAAACCCTGA GCCAGA[G/T]GAG TGAGATTCCC
AGAGCAGGAG AC 3′

A significantly greater proportion of patients diagnosed as having a myeloproliferative disorder (MDS) were homozygous for the G SNP (“the G/G genotype”) compared to the SNP hemizygotes (“the G/T genotype”) and the homozygous wildtypes (“the T/T genotype”). It was further discovered that the G/G genotype was associated with shorter complete remission duration compared to the T/T genotype.

### Sample Collection and Preparation

The methods and compositions of this invention may be used to detect polymorphisms in the EPO promoter using a biological sample obtained from an individual. The nucleic acid may be isolated from the sample according to any methods well known to those of skill in the art. Examples include tissue samples or any cell-containing or acellular bodily fluid. Biological samples may be obtained by standard procedures and may be used immediately or stored, under conditions appropriate for the type of biological sample, for later use.

Methods of obtaining test samples are well known to those of skill in the art and include, but are not limited to, aspirations, tissue sections, drawing of blood or other fluids, surgical or needle biopsies, and the like. The test sample may be obtained from an individual or patient diagnosed as having a myeloproliferative disorder or suspected of being afflicted with a myeloproliferative disorder. The test sample may be a cell-containing liquid or a tissue. Samples may include, but are not limited to, amniotic fluid, biopsies, blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Samples may also be processed, such as sectioning of tissues, fractionation, purification, or cellular organelle separation.

If necessary, the sample may be collected or concentrated by centrifugation and the like. The cells of the sample may be subjected to lysis, such as by treatments with enzymes, heat, surfactants, ultrasonication, or a combination thereof. The lysis treatment is performed in order to obtain a sufficient amount of nucleic acid derived from the individual's cells to detect using polymerase chain reaction.

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70° C. until thawed and used. “Fresh” plasma or serum should be refrigerated or maintained on ice until used, with nucleic acid (e.g., RNA, DNA or total nucleic acid) extraction being performed as soon as possible. Exemplary methods are described below.

Blood can be drawn by standard methods into a collection tube, typically siliconized glass, either without anticoagulant for preparation of serum, or with EDTA, sodium citrate, heparin, or similar anticoagulants for preparation of plasma. If preparing plasma or serum for storage, although not an absolute requirement, is that plasma or serum is first fractionated from whole blood prior to being frozen. This reduces the burden of extraneous intracellular RNA released from lysis of frozen and thawed cells which might reduce the sensitivity of the amplification assay or interfere with the amplification assay through release of inhibitors to PCR such as porphyrins and hematin. “Fresh” plasma or serum may be fractionated from whole blood by centrifugation, using gentle centrifugation at 300-800 times gravity for five to ten minutes, or fractionated by other standard methods. High centrifugation rates capable of fractionating out apoptotic bodies should be avoided.

### Nucleic Acid Extraction and Amplification

The nucleic acid to be amplified may be from a biological sample such as an organism, cell culture, tissue sample, and the like. The biological sample can be from a subject which includes any animal, preferably a mammal. A preferred subject is a human, which may be a patient presenting to a medical provider for diagnosis or treatment of a disease. The volume of plasma or serum used in the extraction may be varied dependent upon clinical intent, but volumes of 100 μL to one milliliter of plasma or serum are usually sufficient.

Various methods of extraction are suitable for isolating the nucleic acid. Suitable methods include phenol and chloroform extraction. See Maniatis et al., Molecular Cloning, A Laboratory Manual, 2d, Cold Spring Harbor Laboratory Press, page 16.54 (1989). Numerous commercial kits also yield suitable DNA and RNA including, but not limited to, QIAamp™ mini blood kit, Agencourt Genfind™, Roche Cobas® Roche MagNA Pure® or phenol:chloroform extraction using Eppendorf Phase Lock Gels®, and the NucliSens extraction kit (Biomerieux, Marcy l'Etoile, France). In other methods, mRNA may be extracted from patient blood/bone marrow samples using MagNA Pure LC mRNA HS kit and Mag NA Pure LC Instrument (Roche Diagnostics Corporation, Roche Applied Science, Indianapolis, Ind.).

Nucleic acid extracted from tissues, cells, plasma or serum can be amplified using nucleic acid amplification techniques well know in the art. Many of these amplification methods can also be used to detect the presence of mutations simply by designing oligonucleotide primers or probes to interact with or hybridize to a particular target sequence in a specific manner. By way of example, but not by way of limitation, these techniques can include the polymerase chain reaction (PCR), reverse transcriptase polymerase chain reaction (RT-PCR), nested PCR, ligase chain reaction. See Abravaya, K., et al., Nucleic Acids Research, 23:675-682, (1995), branched DNA signal amplification, Urdea, M. S., et al., AIDS, 7 (suppl 2):S11-S 14, (1993), amplifiable RNA reporters, Q-beta replication, transcription-based amplification, boomerang DNA amplification, strand displacement activation, cycling probe technology, isothermal nucleic acid sequence based amplification (NASBA). See Kievits, T. et al., J Virological Methods, 35:273-286, (1991), Invader Technology, or other sequence replication assays or signal amplification assays. These methods of amplification each described briefly below and are well-known in the art.

Some methods employ reverse transcription of RNA to cDNA. As noted, the method of reverse transcription and amplification may be performed by previously published or recommended procedures, which referenced publications are incorporated herein by reference in their entirety. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus Thermophilus. For example, one method, but not the only method, which may be used to convert RNA extracted from plasma or serum to cDNA is the protocol adapted from the Superscript II Preamplification system (Life Technologies, GIBCO BRL, Gaithersburg, Md.; catalog no. 18089-011), as described by Rashtchian, A., PCR Methods Applic., 4:S83-S91, (1994).

PCR is a technique for making many copies of a specific template DNA sequence. The reaction consists of multiple amplification cycles and is initiated using a pair of primer sequences that hybridize to the 5′ and 3′ ends of the sequence to be copied. The amplification cycle includes an initial denaturation, and typically up to 50 cycles of annealing, strand elongation and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan, et al., J of Clin Micro, 33(3):556-561 (1995). Briefly, a PCR reaction mixture includes two specific primers, dNTPs, approximately 0.25 U of Taq polymerase, and 1×PCR Buffer.

LCR is a method of DNA amplification similar to PCR, except that it uses four primers instead of two and uses the enzyme ligase to ligate or join two segments of DNA. LCR can be performed as according to Moore et al., J Clin Micro, 36(4):1028-1031 (1998). Briefly, an LCR reaction mixture contains two pair of primers, dNTP, DNA ligase and DNA polymerase representing about 90 μA, to which is added 100 μl of isolated nucleic acid from the target organism. Amplification is performed in a thermal cycler (e.g., LCx of Abbott Labs, Chicago, Ill.).

TAS is a system of nucleic acid amplification in which each cycle is comprised of a cDNA synthesis step and an RNA transcription step. In the cDNA synthesis step, a sequence recognized by a DNA-dependent RNA polymerase (i.e., a polymerase-binding sequence or PBS) is inserted into the cDNA copy downstream of the target or marker sequence to be amplified using a two-domain oligonucleotide primer. In the second step, an RNA polymerase is used to synthesize multiple copies of RNA from the cDNA template. Amplification using TAS requires only a few cycles because DNA-dependent RNA transcription can result in 10-1000 copies for each copy of cDNA template. TAS can be performed according to Kwoh et al., PNAS, 86:1173-7 (1989). Briefly, extracted RNA is combined with TAS amplification buffer and bovine serum albumin, dNTPs, NTPs, and two oligonucleotide primers, one of which contains a PBS. The sample is heated to denature the RNA template and cooled to the primer annealing temperature. Reverse transcriptase (RT) is added the sample incubated at the appropriate temperature to allow cDNA elongation. Subsequently T7 RNA polymerase is added and the sample is incubated at 37° C. for approximately 25 minutes for the synthesis of RNA. The above steps are then repeated. Alternatively, after the initial cDNA synthesis, both RT and RNA polymerase are added following a 1 minute 100° C. denaturation followed by an RNA elongation of approximately 30 minutes at 37° C. TAS can be also be performed on solid phase as according to Wylie et al., J Clin Micro, 36(12):3488-3491 (1998). In this method, nucleic acid targets are captured with magnetic beads containing specific capture primers. The beads with captured targets are washed and pelleted before adding amplification reagents which contains amplification primers, dNTP, NTP, 2500 U of reverse transcriptase and 2500 U of T7 RNA polymerase. A 100 μA TMA reaction mixture is placed in a tube, 200 μA oil reagent is added and amplification is accomplished by incubation at 42° C. in a waterbath for one hour.

NASBA is a transcription-based amplification method which amplifies RNA from either an RNA or DNA target. NASBA is a method used for the continuous amplification of nucleic acids in a single mixture at one temperature. For example, for RNA amplification, avian myeloblastosis virus (AMV) reverse transcriptase, RNase H and T7 RNA polymerase are used. This method can be performed as according to Heim, et al., Nucleic Acids Res., 26(9):2250-2251 (1998). Briefly, an NASBA reaction mixture contains two specific primers, dNTP, NTP, 6.4 U of AMV reverse transcriptase, 0.08 U of Escherichia coli Rnase H, and 32 U of T7 RNA polymerase. The amplification is carried out for 120 min at 41° C. in a total volume of 20 μl.

In a related method, self-sustained sequence-replication (3SR) reaction, isothermal amplification of target DNA or RNA sequences in vitro using three enzymatic activities: reverse transcriptase, DNA-dependent RNA polymerase and Escherichia coli ribonuclease H. This method may be modified from a 3-enzyme system to a 2-enzyme system by using human immunodeficiency virus (HIV)-1 reverse transcriptase instead of avian myeloblastosis virus (AMV) reverse transcriptase to allow amplification with T7 RNA polymerase but without E. coli ribonuclease H. In the 2-enzyme 3SR, the amplified RNA is obtained in a purer form compared with the 3-enzyme 3SR (Gebinoga & Oehlenschlager Eur J Biochem, 235:256-261, 1996).

SDA is an isothermal nucleic acid amplification method. A primer containing a restriction site is annealed to the template. Amplification primers are then annealed to 5′ adjacent sequences (forming a nick) and amplification is started at a fixed temperature. Newly synthesized DNA strands are nicked by a restriction enzyme and the polymerase amplification begins again, displacing the newly synthesized strands. SDA can be performed as according to Walker, et al., PNAS, 89:392-6 (1992). Briefly, an SDA reaction mixture contains four SDA primers, dGTP, dCTP, dTTP, dATP, 150 U of Hinc II, and 5 U of exonuclease-deficient of the large fragment of E. coli DNA polymerase I (exo⁻ Klenow polymerase). The sample mixture is heated 95° C. for 4 minutes to denature target DNA prior to addition of the enzymes. After addition of the two enzymes, amplification is carried out for 120 min. at 37° C. in a total volume of 50° l. Then, the reaction is terminated by heating for 2 min. at 95° C.

The Q-beta replication system uses RNA as a template. Q-beta replicase synthesizes the single-stranded RNA genome of the coliphage Qβ. Cleaving the RNA and ligating in a nucleic acid of interest allows the replication of that sequence when the RNA is replicated by Q-beta replicase (Kramer & Lizardi Trends Biotechnol. 1991 9(2):53-8, 1991).

A variety of amplification enzymes are well known in the art and include, for example, DNA polymerase, RNA polymerase, reverse transcriptase, Q-beta replicase, thermostable DNA and RNA polymerases. Because these and other amplification reactions are catalyzed by enzymes, in a single step assay the nucleic acid releasing reagents and the detection reagents should not be potential inhibitors of amplification enzymes if the ultimate detection is to be amplification based. Amplification methods suitable for use with the present methods include, for example, strand displacement amplification, rolling circle amplification, primer extension preamplification, or degenerate oligonucleotide PCR (DOP). These methods of amplification are well known in the art and each described briefly below.

In suitable embodiments, PCR is used to amplify a target or marker sequence of interest. The skilled artisan is capable of designing and preparing primers that are appropriate for amplifying a target or marker sequence. The length of the amplification primers depends on several factors including the nucleotide sequence identity and the temperature at which these nucleic acids are hybridized or used during in vitro nucleic acid amplification. The considerations necessary to determine a preferred length for an amplification primer of a particular sequence identity are well-known to a person of ordinary skill. For example, the length of a short nucleic acid or oligonucleotide can relate to its hybridization specificity or selectivity.

For analyzing SNPs and other variant nucleic acids, it may be appropriate to use oligonucleotides specific for alternative alleles. Such oligonucleotides which detect single nucleotide variations in target sequences may be referred to by such terms as “allele-specific probes”, or “allele-specific primers”. The design and use of allele-specific probes for analyzing polymorphisms is described in, e.g., Mutation Detection A Practical Approach, ed. Cotton et al. Oxford University Press, 1998; Saiki et al., Nature, 324:163-166 (1986); Dattagupta, EP235,726; and Saiki, WO 89/11548. In one embodiment, a probe or primer may be designed to hybridize to a segment of target DNA such that the SNP aligns with either the 5′ most end or the 3′ most end of the probe or primer.

In some embodiments, the amplification may include a labeled primer, thereby allowing detection of the amplification product of that primer. In particular embodiments, the amplification may include a multiplicity of labeled primers; typically, such primers are distinguishably labeled, allowing the simultaneous detection of multiple amplification products.

In one type of PCR-based assay, an allele-specific primer hybridizes to a region on a target nucleic acid molecule that overlaps a SNP position (e.g., nucleotide position 27 of SEQ ID NO: 1) and only primes amplification of an allelic form to which the primer exhibits perfect complementarity (Gibbs, 1989, Nucleic Acid Res., 17:2427-2448). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target SNP position) because this position is most destabilizing to elongation from the primer (see, e.g., WO 93/22456). Exemplary allele-specific primer sequences for detecting the G polymorphism at SNP position rs1617640 of the EPO promoter are shown in Table 1 below.

TABLE 1
Exemplary Allele-Specific Primers

| | Sequence | |
|---|---|---|
| Description | (5′ to 3′) | SEQ ID NO: |
| Forward WT | GAATCTCACTCA | SEQ ID NO: 2 |
| Allele-Specific Primer | | |
| Forward Mutant | GAATCTCACTCC | SEQ ID NO: 3 |
| Allele-Specific Primer | | |
| Reverse Primer | ATGGCTTCTGGA | SEQ ID NO: 4 |

In a specific embodiment, a primer contains a sequence substantially complementary to a segment of a target SNP-containing nucleic acid molecule except that the primer has a mismatched nucleotide in one of the three nucleotide positions at the 3′-most end of the primer, such that the mismatched nucleotide does not base pair with a particular allele at the SNP site. In one embodiment, the mismatched nucleotide in the primer is the second from the last nucleotide at the 3′-most position of the primer. In another embodiment, the mismatched nucleotide in the primer is the last nucleotide at the 3′-most position of the primer.

In one embodiment, primer or probe is labeled with a fluorogenic reporter dye that emits a detectable signal. While a suitable reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

The present invention also contemplates reagents that do not contain (or that are complementary to) a SNP nucleotide identified herein but that are used to assay one or more SNPs disclosed herein. For example, primers that flank, but do not hybridize directly to a target SNP position provided herein are useful in primer extension reactions in which the primers hybridize to a region adjacent to the target SNP position (i.e., within one or more nucleotides from the target SNP site). During the primer extension reaction, a primer is typically not able to extend past a target SNP site if a particular nucleotide (allele) is present at that target SNP site, and the primer extension product can readily be detected in order to determine which SNP allele is present at the target SNP site. For example, particular ddNTPs are typically used in the primer extension reaction to terminate primer extension once a ddNTP is incorporated into the extension product. Thus, reagents that bind to a nucleic acid molecule in a region adjacent to a SNP site, even though the bound sequences do not necessarily include the SNP site itself, are also encompassed by the present invention.

### Detection of Variant Sequences.

Variant nucleic acids may be amplified prior to detection or may be detected directly during an amplification step (i.e., “real-time” methods). In some embodiments, the target sequence is amplified and the resulting amplicon is detected by electrophoresis. In some embodiments, the specific mutation or variant is detected by sequencing the amplified nucleic acid. In some embodiments, the target sequence is amplified using a labeled primer such that the resulting amplicon is detectably labeled. In some embodiments, the primer is fluorescently labeled.

In one embodiment, detection of a variant nucleic acid, such as a SNP, is performed using the TaqMan® assay, which is also known as the 5′ nuclease assay (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635). The TaqMan® assay detects the accumulation of a specific amplified product during PCR. The TaqMan® assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

TaqMan® primer and probe sequences can readily be determined using the variant and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the variants of the present invention are useful in diagnostic assays for neurodevelopmental disorders and related pathologies, and can be readily incorporated into a kit format. The present invention also includes modifications of the TaqMan® assay well known in the art such as the use of Molecular Beacon probes (U.S. Pat. Nos. 5,118,801 and 5,312,728) and other variant formats (U.S. Pat. Nos. 5,866,336 and 6,117,635).

In an illustrative embodiment, real time PCR is performed using TaqMan® probes in combination with a suitable amplification/analyzer such as the ABI Prism® 7900HT Sequence Detection System. The ABI PRISM® 7900HT Sequence Detection System is a high-throughput real-time PCR system that detects and quantitates nucleic acid sequences. Briefly, TaqMan® probes specific for the amplified target or marker sequence are included in the PCR amplification reaction. These probes contain a reporter dye at the 5′ end and a quencher dye at the 3′ end. Probes hybridizing to different target or marker sequences are conjugated with a different fluorescent reporter dye. During PCR, the fluorescently labeled probes bind specifically to their respective target or marker sequences; the 5′ nuclease activity of Taq polymerase cleaves the reporter dye from the probe and a fluorescent signal is generated. The increase in fluorescence signal is detected only if the target or marker sequence is complementary to the probe and is amplified during PCR. A mismatch between probe and target greatly reduces the efficiency of probe hybridization and cleavage. The ABI Prism 7700HT or 7900HT Sequence detection System measures the increase in fluorescence during PCR thermal cycling, providing “real time” detection of PCR product accumulation. Real time detection on the ABI Prism 7900HT or 7900HT Sequence Detector monitors fluorescence and calculates Rn during each PCR cycle. The threshold cycle, or Ct value, is the cycle at which fluorescence intersects the threshold value. The threshold value is determined by the sequence detection system software or manually.

Exemplary allele-specific probe sequences for detecting the G polymorphism at SNP position rs1617640 of the EPO promoter in a TaqMan assay are shown in Table 2 below.

TABLE 2
Exemplary Allele-Specific TaqMan ® Probes

| | Sequence | |
|---|---|---|
| Description | (5′ to 3′) | SEQ ID NO: |
| TaqMan WT (G902) | TCACTCATCTGGC | SEQ ID NO: 5 |
| Allele-Specific Probe | | |
| TaqMan Mutant (A902) | TCACTCCTCTGGC | SEQ ID NO: 6 |
| Allele-Specific Probe | | |

Other methods of probe hybridization detected in real time can be used for detecting amplification a target or marker sequence flanking a tandem repeat region. For example, the commercially available MGB Eclipse™ probes (Epoch Biosciences), which do not rely on a probe degradation can be used. MGB Eclipse™ probes work by a hybridization-triggered fluorescence mechanism. MGB Eclipse™ probes have the Eclipse™Dark Quencher and the MGB positioned at the 5′-end of the probe. The fluorophore is located on the 3′-end of the probe. When the probe is in solution and not hybridized, the three dimensional conformation brings the quencher into close proximity of the fluorophore, and the fluorescence is quenched. However, when the probe anneals to a target or marker sequence, the probe is unfolded, the quencher is moved from the fluorophore, and the resultant fluorescence can be detected.

Oligonucleotide probes can be designed which are between about 10 and about 100 nucleotides in length and hybridize to the amplified region. Oligonucleotides probes are preferably 12 to 70 nucleotides; more preferably 15-60 nucleotides in length; and most preferably 15-25 nucleotides in length. The probe may be labeled. Amplified fragments may be detected using standard gel electrophoresis methods. For example, in preferred embodiments, amplified fractions are separated on an agarose gel and stained with ethidium bromide by methods known in the art to detect amplified fragments.

Another suitable detection methodology involves the design and use of bipartite primer/probe combinations such as Scorpion™ probes. These probes perform sequence-specific priming and PCR product detection is achieved using a single molecule. Scorpion™ probes comprise a 3′ primer with a 5′ extended probe tail comprising a hairpin structure which possesses a fluorophore/quencher pair. The probe tail is “protected” from replication in the 5′ to 3′ direction by the inclusion of hexethlyene glycol (HEG) which blocks the polymerase from replicating the probe. The fluorophore is attached to the 5′ end and is quenched by a moiety coupled to the 3′ end. After extension of the Scorpion™ primer, the specific probe sequence is able to bind to its complement within the extended amplicon thus opening up the hairpin loop. This prevents the fluorescence from being quenched and a signal is observed. A specific target is amplified by the reverse primer and the primer portion of the Scorpion™, resulting in an extension product. A fluorescent signal is generated due to the separation of the fluorophore from the quencher resulting from the binding of the probe element of the Scorpion™ to the extension product. Such probes are described in Whitcombe et al., Nature Biotech 17: 804-807 (1999).

### Determining Prognosis

Provided herein are methods of using the SNP/genotype status at SNP position rs1617640 of the EPO promoter in a test sample from a patient, alone or in conjunction with clinical factors, in determining the prognosis for a patient having a myeloproliferative disease. Other genes or loci may also be characterized, such as genes or loci associated with cellular proliferation, particularly proliferation of blood cells. Other genes or loci of potential interest those known to be involved in leukemias. Genes of potential interest include, but are not limited to, those that regulate the expression of cytokines, growth factors, angiogenic factors, oncogenes and proteins associated with the development of blood cells. Such genes may include ras, p53, WT1, ETV6 (TEL), MLL, CBP and AC133. Furthermore, chromosomal translocations may also be investigated, such as t(11;16), t(9:21), t(10;21) and der(6:19). Examples of genes differentially expressed in MDS are known in the art, such as described in Miyazato et al., Blood, (2001) 98:422-427.

In some embodiments, prognosis may be a prediction of the likelihood that a patient will survive for a particular period of time, or the prognosis is a prediction of how long a patient may live, or the prognosis is the likelihood that a patent will recover from a disease or disorder. There are many ways that prognosis can be expressed. For example prognosis can be expressed in terms of complete remission rates (CR), overall survival (OS) which is the amount of time from entry to death, remission duration, which is the amount of time from remission to relapse or death.

In certain embodiments the SNP status of rs1617640 (i.e., G/G, T/T, or G/T) is used as an indicator of an prognosis, for example, in MDS. For example, patients having the G/G genotype (i.e., homozygous for G polymorphism) are identified as likely to have a shorter complete remission duration relative to those having the G/T or T/T genotypes.

In certain embodiments, the prognosis of MDS patients can be correlated to the clinical outcome of the disease using the SNP status of rs1617640 and other clinical factors. Simple algorithms have been described and are readily adapted to this end. The approach by Giles et. al., British Journal of Haemotology, 121:578-585, is exemplary. As in Giles et al., associations between categorical variables (e.g., proteasome activity levels and clinical characteristics) can be assessed via crosstabulation and Fisher's exact test. Unadjusted survival probabilities can be estimated using the method of Kaplan and Meier. The Cox proportional hazards regression model also can be used to assess the ability of patient characteristics (such as proteasome activity levels) to predict survival, with ‘goodness of fit’ assessed by the Grambsch-Therneau test, Schoenfeld residual plots, martingale residual plots and likelihood ratio statistics (see Grambsch, 1995; Grambsch et al, 1995).

In some embodiments of the invention, multiple prognostic factors, including the SNP status of rs1617640, are considered when determining the prognosis of a patient. For example, the prognosis of an MDS patient may be determined based on SNP status of rs1617640 and one or more prognostic factors selected from the group consisting of cytogenetics, performance status, AHD (antecedent hematological disease), and age. In certain embodiments, other prognostic factors may be combined with the SNP status of rs1617640 in the algorithm to determine prognosis with greater accuracy.

### Risk Association

To determine the association of a particular genotype with a disease or disease progression, the genotype of subjects with the disease is compared to the genotype of subjects without the disease. For many diseases, it is preferable to also compare the disease genotype with the genotypes from subjects having a related or similar disease so as to better identify genotypes that are specific for the disease of interest.

For example, the genotype of subjects having MDS can be compared to normal subjects, preferably matched as described above. Further, the MDS genotypes can be compared to subjects having AML.

Once the genotypes of each group are known, the risk of developing a disease, such as MDS, or the duration of remission, can be determined statistically. One such method for calculating the risk is using odds ratios (OR). This widely used statistic compares the retrospective/posterior odds of exposure to a given risk factor in two groups of individuals. The OR can be manually calculated using contingency tables for each SNP, as shown in Table 2.

TABLE 3
Odds Ratios

| | Genotype 1 | Genotype 2 |
|---|---|---|
| Disease | A | B |
| Control | C | D |

Odds of Exposure in Disease=A×(A+B)/B×(A+B)=A/B

Odds of Exposure in Controls=C×(C+D)/D×(C+D)=C/D

Odds Ratio=(A/B)/(C/D)=(A×D)/(B×C)

To determine if the OR is statistically significant, a confidence interval (CI) of 95% is generally set, as follows.

95% CI of ln(OR)=ln(OR)+−1.96(1/A+1/B+1/C+1/D)0.5

More commonly, a statistical software package may be used, particularly when more than one SNP is being evaluated. Numerous such software packages are available, both commercially and via publicly available websites, such as the Genetic Power Calculator, Purcell S, et al.(2003) Bioinformatics, 19(1):149-150.

### Kits

Also provided are kits comprising the peptides described herein. The kits may be prepared for practicing the methods described herein. Typically, the kits include at least one component or a packaged combination of components useful for practicing a method. The kits may include some or all of the components necessary to practice a method disclosed herein. Typically, the kits include at least one peptide probe in at least one container. These components may included, inter alia, nucleic acid probes, nucleic acid primers for amplification of the region of interest, buffers, instructions for use, and the like.

### Example 1

To investigate the association between the genotype of EPO SNP rs1617640 with various leukemias, the following patient populations were genotyped: MDS patients (n=187), AML patients (n=257), ALL patients (n=106), CLL patients (n=97), CML patients (n=353), and healthy controls (n=95).

As detailed in Table 4, the MDS and ALL patient populations showed the highest proportion of individuals with the G/G genotype and were significantly above control levels, demonstrating that the G/G genotype is a risk factor for at least these diseases. The AML, CLL, and CML patients, while demonstrating an elevated proportion of the G/G genotype, did not reach statistical significance in this study. When all leukemia patients were considered together, rather than being stratified based on leukemia subtype, the odds of having the G/G genotype were higher than the control population. This increased statistical power indicates that the G/G genotype is a risk factor for developing leukemia.

TABLE 4
Distribution of rs1617640 EPO SNP Genotype in Normal Control
Subjects and Patients with Hematologic Diseases

| | | | | | | P-value | P-value |
|---|---|---|---|---|---|---|---|
| | | | | | | (vs. | (vs. all |
| | | EPO SNP Genotype | | | | normal | leukemia |
| Diagnosis | | G/G | G/T | T/T | Total | controls)^{a} | samples)^{a} |
| Normal | n | 6 | 41 | 48 | 95 | | 0.02 |
| | % | 6.3 | 43.2 | 50.5 | | | |
| MDS | n | 47 | 73 | 67 | 187 | <0.001 | <0.001 |
| | % | 25.1 | 39 | 35.8 | | | |
| ALL | n | 14 | 62 | 30 | 106 | 0.03 | 0.61 |
| | % | 13.2 | 58.5 | 28.3 | | | |
| AML | n | 32 | 115 | 110 | 257 | 0.1 | 0.21 |
| | % | 12.5 | 44.8 | 42.8 | | | |
| CLL | n | 11 | 34 | 52 | 97 | 0.22 | 0.31 |
| | % | 11.3 | 35.1 | 53.6 | | | |
| CML | n | 44 | 173 | 136 | 353 | 0.09 | 0.1 |
| | % | 12.5 | 49 | 38.5 | | | |
| Total | n | 154 | 498 | 443 | 1095 | | |
| | % | 14.1 | 45.5 | 40.5 | 100 | | |

^{a}Fisher's exact test.

The odds ratio (OR) for having the G/G genotype in MDS patients when compared with normal control was 4.98 with 95% confidence interval (CI) of 2.04-12.13 (P=0.0002). Using the odds ratio calculation, the relationship between the G/G genotype and ALL falls just short of statistical significance in this study. However, when statistical power is increased by considering all leukemia patients together, the odds ratio versus control is significant for the G/G genotype. There was no significant difference in having the G/T or the T/T genotypes between the patients with any leukemia and the control group.

TABLE 5
Odds and Risk Ratios for EPO SNP rs1617640 Genotypes
in Patients with Hematologic Diseases

| | |
|---|---|
| Odds Ratio | Relative Risk |

| | OR | 95% CI | RR | 95% CI |
|---|---|---|---|---|
| MDS vs. ALL | | | | |
| G/G | 2.2 | 1.15-4.24 | 1.28 | 1.07-1.52 |
| G/T | 0.45 | 0.28-0.74 | 0.75 | 0.62-0.90 |
| T/T | 1.41 | 0.84-2.37 | 1.13 | 0.95-1.34 |
| MDS vs. AML | | | | |
| G/G | 2.36 | 1.44-3.88 | 1.55 | 1.24-1.94 |
| G/T | 0.79 | 0.54-1.16 | 0.87 | 0.70-1.09 |
| T/T | 0.75 | 0.56-1.10 | 0.84 | 0.67-1.06 |
| MDS vs. CLL | | | | |
| G/G | 2.62 | 1.29-5.33 | 1.31 | 1.11-1.54 |
| G/T | 1.19 | 0.71-1.98 | 1.06 | 0.89-1.25 |
| T/T | 0.48 | 0.29-0.80 | 0.77 | 0.64-0.93 |
| MDS vs. CML | | | | |
| G/G | 2.36 | 1.49-3.72 | 1.66 | 1.30-2.11 |
| G/T | 0.67 | 0.47-0.96 | 0.77 | 0.60-0.97 |
| T/T | 0.89 | 0.62-1.29 | 0.93 | 0.73-1.18 |
| MDS vs. normal | | | | |
| G/G | 4.98 | 2.04-12.13 | 1.45 | 1.26-1.67 |
| G/T | 0.84 | 0.51-1.39 | 0.94 | 0.79-1.12 |
| T/T | 0.55 | 0.33-0.90 | 0.81 | 0.68-0.97 |
| MDS vs. other | | | | |
| leukemias | | | | |
| G/G | 2.37 | 1.60-3.50 | 1.93 | 1.46-2.56 |
| G/T | 0.72 | 0.52-0.99 | 0.76 | 0.58-0.99 |
| T/T | 0.83 | 0.59-1.15 | 0.86 | 0.65-1.12 |
| ALL vs. AML | | | | |
| G/G | 1.07 | 0.55-2.10 | 1.05 | 0.66-1.68 |
| G/T | 1.74 | 1.10-2.75 | 1.48 | 1.07-2.05 |
| T/T | 0.53 | 0.32-0.86 | 0.63 | 0.44-0.91 |
| ALL vs. CLL | | | | |
| G/G | 1.19 | 0.51-2.76 | 1.08 | 0.74-1.58 |
| G/T | 2.61 | 1.48-4.61 | 1.57 | 1.20-2.06 |
| T/T | 0.34 | 0.19-0.61 | 0.58 | 0.42-0.80 |
| ALL vs. CML | | | | |
| G/G | 1.07 | 0.56-2.04 | 1.05 | 0.64-1.72 |
| G/T | 1.47 | 0.95-2.27 | 1.34 | 0.96-1.89 |
| T/T | 0.63 | 0.39-1.01 | 0.70 | 0.48-1.02 |
| ALL vs. normal | | | | |
| G/G | 2.26 | 0.83-6.13 | 1.38 | 1.00-1.90 |
| G/T | 1.86 | 1.06-3.25 | 1.34 | 1.02-1.76 |
| T/T | 0.39 | 0.22-0.69 | 0.62 | 0.46-0.85 |
| ALL vs. other | | | | |
| leukemias | | | | |
| G/G | 0.86 | 0.48-1.56 | 0.88 | 0.51-1.50 |
| G/T | 1.78 | 1.18-2.68 | 1.67 | 1.16-2.41 |
| T/T | 0.57 | 0.37-0.89 | 0.60 | 0.40-0.90 |
| AML vs. CLL | | | | |
| G/G | 1.11 | 0.54-2.30 | 1.03 | 0.85-1.24 |
| G/T | 1.50 | 0.92-2.44 | 1.11 | 0.98-1.26 |
| T/T | 0.65 | 0.41-1.04 | 0.89 | 0.78-1.01 |
| AML vs. CML | | | | |
| G/G | 1.00 | 0.61-1.62 | 1.00 | 0.75-1.32 |
| G/T | 0.84 | 0.61-1.16 | 0.91 | 0.75-1.09 |
| T/T | 1.19 | 0.86-1.66 | 1.11 | 0.92-1.33 |
| AML vs. normal | | | | |
| G/G | 2.11 | 0.85-5.22 | 1.18 | 1.01-1.37 |
| G/T | 1.07 | 0.66-1.71 | 1.02 | 0.90-1.16 |
| T/T | 0.73 | 0.46-1.17 | 0.92 | 0.81-1.05 |
| AML vs. other | | | | |
| leukemias | | | | |
| G/G | 0.77 | 0.51-1.17 | 0.82 | 0.59-1.14 |
| G/T | 0.95 | 0.71-1.26 | 0.96 | 0.78-1.19 |
| T/T | 1.20 | 0.90-1.60 | 1.15 | 0.93-1.42 |
| CLL vs. CML | | | | |
| G/G | 0.90 | 0.44-1.81 | 0.92 | 0.52-1.61 |
| G/T | 0.56 | 0.35-0.90 | 0.63 | 0.44-0.92 |
| T/T | 1.84 | 1.17-2.90 | 1.61 | 1.13-2.29 |
| CLL vs. normal | | | | |
| G/G | 1.90 | 0.67-5.36 | 1.32 | 0.90-1.93 |
| G/T | 0.71 | 0.40-1.27 | 0.84 | 0.62-1.14 |
| T/T | 1.13 | 0.64-1.99 | 1.06 | 0.80-1.41 |
| CLL vs. other | | | | |
| leukemias | | | | |
| G/G | 0.72 | 0.37-1.37 | 0.74 | 0.40-1.35 |
| G/T | 0.61 | 0.40-0.95 | 0.64 | 0.43-0.95 |
| T/T | 1.89 | 1.24-2.87 | 1.77 | 1.21-2.58 |
| CML vs. normal | | | | |
| G/G | 2.11 | 0.87-5.12 | 1.13 | 1.01-1.27 |
| G/T | 1.27 | 0.80-2.00 | 1.05 | 0.95-1.16 |
| T/T | 0.61 | 0.39-0.97 | 0.90 | 0.81-1.00 |
| CML vs. other | | | | |
| leukemias | | | | |
| G/G | 0.74 | 0.51-1.09 | 0.82 | 0.63-1.07 |
| G/T | 1.23 | 0.95-1.59 | 1.14 | 0.97-1.35 |
| T/T | 0.94 | 0.72-1.22 | 0.96 | 0.81-1.14 |
| Leukemia vs. | | | | |
| normal | | | | |
| G/G | 2.58 | 1.11-6.00 | 1.06 | 1.02-1.10 |
| G/T | 1.11 | 0.73-1.70 | 1.01 | 0.97-1.05 |
| T/T | 0.64 | 0.42-0.97 | 0.96 | 0.92-1.00 |
| Leukemias | | | | |
| (except MDS) | | | | |
| vs. normal | | | | |
| G/G | 2.10 | 0.90-4.94 | 1.06 | 1.01-1.12 |
| G/T | 1.18 | 0.77-1.81 | 1.02 | 0.97-1.06 |
| T/T | 0.66 | 0.43-1.01 | 0.96 | 0.91-1.00 |

Clinical and follow up data was available on 112 MDS patients and 186 AML patients. There was no correlation between EPO promoter genotype with response to therapy or overall survival in MDS or AML. In the MDS group, the G/G genotype was significantly associated with shorter complete remission duration as compared with patients with the T/T genotype (P=0.03). Also no correlation was found between EPO genotype and cytogenetic abnormalities, performance status or other laboratory parameters.

### Example 2

To investigate the association between the genotype of EPO SNP rs1617640 with various leukemias, the following patient populations were genotyped: suspected myeloproliferative disorder (MPD) patients (n=48) and AML patients (n=70). 49 normal patient samples were also tested.

### Materials and Methods

Genomic DNA was extracted from whole blood and plasma samples. DNA extraction from whole blood used BioRobot EZ1; DNA extraction from plasma used Biomerieux NucliSens EasyMAG Nucleic Acid Purification System.

SNP detection used PCR primers in combination with TaqMan MGB probes designed to detect the two SNP alleles (G and T). During PCR, each of the MGB probes anneals specifically to its complementary sequence between the forward and reverse primer sites. Detection is achieved with 5′ nuclease chemistry by means of exonuclease cleavage of a 5′ allele-specific dye label which generates the permanent assay signal. The EPO forward and reverse primers (SEQ ID NO: 7 and 8, respectively) and the EPO-G and EPO-T TaqMan MGB probes (SEQ ID NO: 9 AND 10, respectively) are listed below.

SEQ ID NO: 7
GGGCTGGGATTTACAGCTAA
SEQ ID NO: 8
CCAGCTAGTCTTGGTCTCCTG
SEQ ID NO: 9
vic-TGAGCCAGAGGAGTGA-MGBNFQ
SEQ ID NO: 10
6FAM-CTGAGCCAGATGAGTGA-MGBNFQ

Genotyping master mix containing enzymes, buffers, primers, probes, and dNTPs was prepared according to table 7 below.

TABLE 7
Genotyping Master Mix Composition

| Components | Final Concentration | |
|---|---|---|
| 2x Reaction Buffer | 1x | |
| dNTP | 250 | μM |
| EPO Forward primer | 0.4 | μM |
| EPO Reverse primer | 0.4 | μM |
| T Fam probe | 0.1 | μM |
| G Vic probe | 0.1 | μM |
| FastStart Taq | 1.25 | U |

DNA template from each sample was added to a portion of the master mix, and the reaction mixture was amplified using two-step PCR in an ABI 7900HT Sequence Detection System for 50 cycles (95° C. for 15 seconds, 60° C. for 1.5 minutes). The results of the amplification reaction were then read and analyzed to determine the amplified alleles.

### Results

Both the AML and suspected MDS patient populations had a greater percentage of G/G homozygotes than the normal population tested. The results are summarized in Table 8 below.

TABLE 8
Summary of Allele Frequency in
AML and Suspected MPD Patients

| Genotype | AML | % | Susp. MPD | % | Normal | % |
|---|---|---|---|---|---|---|
| G/G | 17 | 24.29% | 7 | 14.58% | 3 | 6.12% |
| G/T | 33 | 47.14% | 17 | 35.42% | 23 | 46.94% |
| T/T | 20 | 28.57% | 24 | 50.00% | 23 | 46.94% |
| TOTAL | 70 | | 48 | | 48 | |

The normal population diversity among various ethnic groups from NCBI SNPweb and the normal patients sampled in this study is shown below in table 9.

TABLE 9
Normal Population Diversity

| Ethnic Group | Sample # | G/G | G/T | T/T | Source |
|---|---|---|---|---|---|
| European | 120 | 15.00% | 48.30% | 36.70% | NCBI SNPweb |
| Asian | 90 | 4.40% | 35.60% | 60.00% | NCBI SNPweb |
| Sub-Saharan | 120 | 15.00% | 35.00% | 50.00% | NCBI SNPweb |
| African | | | | | |
| Study Normal | 49 | 6.12% | 46.94% | 46.94% | Validation |
| | | | | | Study |

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

1. A method of determining a prognosis for a subject diagnosed with leukemia comprising:
a) determining the zygosity status of the subject at the nucleotide corresponding to SNP1617640 in the erythropoietin gene promoter; and
b) identifying the subject as having a poor prognosis when the zygosity status is homozygous G/G.

2. The method of claim 1, wherein the leukemia is selected from the group consisting of myelodysplastic syndrome (MDS), acute lymphoblastic leukemia (ALL), acute myeloid leukemia (AML), chronic lymphocytic leukemia (CLL), and chronic myeloid leukemia (CML).

3. The method of claim 2, wherein the leukemia is MDS or ALL.

4. The method of claim 1, wherein the zygosity status is determined by assessing subject nucleic acid obtained from a biological sample.

5. The method of claim 4, wherein the biological sample is whole blood, blood serum, or plasma.

6. The method of claim 1, wherein the poor prognosis is selected from the group consisting of shorter survival, shorter complete remission duration, and shorter event-free survival.

7. The method of claim 6, wherein the poor prognosis is shorter complete remission duration.

8. The method of claim 1, further comprising assessing clinical factors and using the zygosity status and the clinical factors for determining the prognosis.

9. The method of claim 1, wherein the zygosity status is determined using a technique selected from the group consisting of nucleic acid sequencing, probe hybridization, and a primer extension reaction.

10. A method of identifying a subject at risk of developing leukemia comprising:
a) determining the zygosity status of the subject at the nucleotide corresponding to SNP1617640 in the erythropoietin gene promoter; and
b) identifying the subject as having increased risk of leukemia when the zygosity status is homozygous G/G.

11. The method of claim 10, wherein the leukemia is selected from the group consisting of myelodysplastic syndrome (MDS), acute lymphoblastic leukemia (ALL), acute myeloid leukemia (AML), chronic lymphocytic leukemia (CLL), and chronic myeloid leukemia (CML).

12. The method of claim 11, wherein the leukemia is MDS or ALL.

13. The method of claim 10, wherein the zygosity status is determined by assessing subject nucleic acid obtained from a biological sample.

14. The method of claim 13, wherein the biological sample is whole blood, blood serum, or plasma.

15. The method of claim 10, further comprising assessing clinical factors and using the zygosity status and the clinical factors for determining the prognosis.

16. The method of claim 10, wherein the zygosity status is determined using a technique selected from the group consisting of nucleic acid sequencing, probe hybridization, and a primer extension reaction.
